(19) United States
(12) Patent Application Publication
(10) Pub. No.: US 2018/0256237 A1
(43) Pub. Date: Sep. 13, 2018

(54) CATHETER SLEEVE FOR CRYOTHERAPY SYSTEM

(71) Applicant: CSA Medical, Inc., Lexington, MA (US)

(72) Inventors: Wei L. Fan, Boston, MA (US); Brian M. Hanley, Reading, MA (US); Sean A. McDermott, Weymouth, MA (US); John P. O'Connor, Andover, MA (US)

(21) Appl. No.: 15/914,510

(22) Filed: Mar. 7, 2018

Related U.S. Application Data
(60) Provisional application No. 62/469,066, filed on Mar. 9, 2017.

(51) Int. Cl. A61B 18/02; A61B 1/018

(52) U.S. Cl. CPC A61B 18/0218; A61B 2017/0034; A61B 1/018

(57) ABSTRACT

The present disclosure relates generally to the field of cryotherapy. In particular, the present disclosure relates to catheter sleeves for cryotherapy systems that inhibit and remove build-up and backflow of undesired material in the working channel of endoscopes during cryotherapy treatments.

### PRIORITY

This application claims the benefit of priority under 35 U.S.C. § 119 to U.S. Provisional Patent Application Ser. No. 62/469,066, filed Mar. 9, 2017, which is incorporated by reference herein in its entirety and for all purposes.

### FIELD

The present disclosure relates generally to the field of cryotherapy. In particular, the present disclosure relates to devices and methods to inhibit and remove build-up and backflow of undesired material within the endoscope working channel of a cryotherapy system. More specifically, the present disclosure relates to a catheter sleeve configured to eliminate the gap-space between a cryogen delivery catheter and endoscope working channel of a cryotherapy system.

### BACKGROUND

As a cryotherapy example, cryoablation is a surgical procedure in which diseased, damaged or otherwise undesirable tissue (collectively referred to herein as “target tissue” and/or “treatment region”) may be destroyed by local delivery of a cryogen spray. These systems along with other cryotherapy systems are typically referred to as cryoablation systems, cryospray systems, cryospray ablation systems, cryosurgery systems, cryosurgery spray systems and/or cryogen spray ablation systems. The term “cryogen” typically refers to any fluid (e.g., gas, liquefied gas or other fluid known to one of ordinary skill in the art) with a sufficiently low boiling point (i.e., below approximately −153° C.) for therapeutically effective use during a cryotherapy procedure. Suitable cryogens may include, for example, liquid argon, liquid nitrogen and liquid helium. Pseudo-cryogens such as liquid carbon dioxide and liquid nitrous oxide that have a boiling temperature above −153° C. but still very low (e.g., −89° C. for liquid N₂O) may also be used.

For example, during operation of a cryospray ablation system, a medical professional (e.g., clinician, technician, physician, surgeon, etc.) directs a cryogen spray onto the surface of a treatment area via a cryogen delivery catheter. The medical professional may target the cryogen spray visually through a video-assisted medical device, such as an endoscope, bronchoscope, colonoscope or ureteroscope. Cryogen spray exits the cryogen delivery catheter at a temperature ranging from 0° C. to −196° C. (for liquid nitrogen), causing the tissue of the treatment area (e.g., target tissue) to freeze or “cryofrost.” As the liquid cryogen exits the cryogen delivery catheter and contacts the treatment area, it converts to a gaseous state with a significant increase in volume. For example, 1 cubic centimeter (cm³) of liquid nitrogen converts to 694 cm³ of nitrogen gas at body temperature.

In some cases, expanding gases may flow into the working channel of the endoscope as a result of a gap-space that typically exists between the outer surface of a catheter and the inner surface of the working channel, either directly as the gases exit from the cryogen delivery catheter or indirectly as the cryogen spray circulates within the body lumen. The accumulation or buildup and backflow of cryogen spray and other undesired materials such as, for example, fluids from the gastrointestinal tract or the airways, gases, and liquids within the working channel of the endoscope, may provide a heat sink effect, which diverts cryotherapy energy away from the treatment area, thereby leading to increased procedure times and/or suboptimal therapeutic results. This effect is often referred to as “weak spray”. It is also possible that the liquids within the working channel may freeze causing issues with motion of the endoscope and prolonged effects of “weak spray”. There is as well a need for or desirability of preventing negative thermal effects to the cryospray by elimination of the gas or fluid buildup within the working channel of an endoscope also may provide protection of sensors, or other features, located on the catheter or within the endoscope assembly from deleterious effects of undesired fluids and help to ensure their accuracy during cryosprays. An example is the use of a thermocouple mounted on the catheter to monitor the temperature of the catheter during a cryospray. Prevention of fluid/gas buildup in the region of the thermocouple would help ensure higher accuracy measurements with more precise and accurate cryogen spray controls.

The negative effects of cryogen spray and any other accumulation may be partially addressed by intermittently clearing the endoscope working channel throughout the course of the medical procedure. However, the additional steps required to remove the accumulated cryogen spray (e.g., removing the endoscope from the patient and/or introducing a cleaning element etc.) may significantly increase both procedure times and the possibility of negative medical outcomes. Accordingly, various advantages may be realized by a cryotherapy system as described herein that inhibits or removes the accumulation or buildup and backflow of cryogen spray and other undesired materials, gases and liquids during a cryotherapy medical procedure.

### SUMMARY

Embodiments of the present disclosure may include a cryogen delivery catheter that may include an elongated shaft having a distal end portion. The catheter may include a sleeve section comprising an outer surface. The outer surface may include at least one first portion with a first diameter and at least one second portion with a second diameter that is greater than the first diameter and that is dimensioned to contact an inner surface of the working channel of the endoscope.

A catheter may include a sleeve section that is a tubular member defining a lumen. The sleeve section may be integral with the shaft. The catheter may include a sleeve section with a first diameter that is the same as an outer diameter of the catheter shaft. The catheter may include a second portion with a radial mid-body band. The second portion may include at least one channeled rib. The second portion may include at least one radial rib. The second portion may include a radial spiral rib. The second portion may include a tapered incline with a slope facing distally. The second portion may include a radial flared end. The second portion may include a planar middle portion that tapers in diameter at both ends to a first diameter of a first portion.

A catheter may include a sleeve section that includes a radial band with a sloped proximal surface and a sloped distal surface. The sleeve section may include a lumen with a tapered diameter. The catheter may include a second portion of an outer surface of the sleeve section where at least the second portion comprises a compressible polymeric material. The sleeve section may comprise a lubricious coating.

A cryotherapy system may include an endoscope. The system may include a cryogen delivery catheter slidably disposed within a working channel of the endoscope. The system may include a catheter sleeve disposed around a distal portion of the cryogen delivery catheter. The catheter sleeve may include an outer surface having a first diameter portion and a second diameter portion. The second diameter portion may have a greater diameter than the first diameter portion and may be configured to contact an inner wall of the working channel. The second diameter portion may be configured to span a gap-space between the distal portion of the cryogen delivery catheter and the working channel of the endoscope. The catheter sleeve may form an interference fit with the distal portion of the cryogen delivery catheter. The catheter sleeve may be adhered to the distal portion of the cryogen delivery catheter. The diameter of the second diameter portion may be greater than or equal to a diameter of the working channel of the endoscope. The second diameter portion may be configured to remove or inhibit build-up of undesired material from the working channel of the endoscope as the delivery catheter slides distally within the working channel. The second diameter portion may be configured to block a distal opening of the working channel of the endoscope. The catheter sleeve may be integrally formed as a part of the distal portion of the cryogen delivery catheter.

A method of maintaining a working channel of an endoscope may include inserting a catheter into the working channel of the endoscope with a distal end portion having a sleeve section comprising an outer surface. The outer surface may include at least one first portion with a first diameter and at least one second portion with a second diameter that is greater than the first diameter and that is dimensioned to contact an inner surface of the working channel of the endoscope. The method may include translating the catheter within the working channel of the endoscope. A method may include shielding at least one sensor mounted on the catheter or within the working channel of the endoscope proximal to the sleeve section, whereby undesired thermal effects due to fluid or gas buildup within the working channel are mitigated. The method may include eliminating undesired gases or fluids from the working channel of the endoscope, whereby negative thermal effects on the strength of cryospray during a cryotherapy procedure are minimized.

### BRIEF DESCRIPTION OF THE DRAWINGS

Non-limiting embodiments of the present disclosure are described by way of example with reference to the accompanying figures, which are schematic and not intended to be drawn to scale. In the figures, each identical or nearly identical component illustrated is typically represented by a single numeral. For purposes of clarity, not every component is labeled in every figure, nor is every component of each embodiment shown where illustration is not necessary to allow those of ordinary skill in the art to understand the disclosure. In the figures:

FIG. 1 illustrates a catheter and sleeve, in accordance with an embodiment of the present disclosure.

FIG. 2 illustrates a catheter sleeve including a radial elongate band, in accordance with an embodiment of the present disclosure.

FIG. 3 illustrates a catheter sleeve including radial V-channel ribs, in accordance with an embodiment of the present disclosure.

FIG. 4 illustrates a catheter sleeve including radial ribs, in accordance with an embodiment of the present disclosure.

FIG. 5 illustrates a catheter sleeve including a radial spiral rib, in accordance with an embodiment of the present disclosure.

FIG. 6 illustrates a catheter sleeve including a radial elongate band, in accordance with another embodiment of the present disclosure.

FIG. 7 illustrates a catheter sleeve including a radial flared end, in accordance with an embodiment of the present disclosure.

FIG. 8 illustrates a catheter sleeve including a tapered incline end, in accordance with an embodiment of the present disclosure.

FIG. 9 illustrates a catheter sleeve including a radial band with a sloped proximal surface and a sloped distal surface, in accordance with an embodiment of the present disclosure.

FIG. 10 illustrates a catheter sleeve including a tapered lumen, in accordance with an embodiment of the present disclosure.

FIG. 11 illustrates a cryotherapy system, including a catheter sleeve of a cryogen delivery catheter within a working channel of an endoscope, in accordance with an embodiment of the present disclosure.

### DETAILED DESCRIPTION

The present disclosure is not limited to the particular embodiments described. The terminology used herein is for the purpose of describing particular embodiments only, and is not intended to be limiting beyond the scope of the appended claims. Unless otherwise defined, all technical terms used herein have the same meaning as commonly understood by one of ordinary skill in the art to which the disclosure pertains.

Although embodiments of the present disclosure are described with specific reference to cryotherapy systems for use within the upper and lower GI tracts and respiratory system, it should be appreciated that such systems and methods may be used in a variety of other body passageways, organs and/or cavities, such as the vascular system, urogenital system, lymphatic system, neurological system and the like.

It is also contemplated that sleeves according to the present disclosure with respect to the embodiments described herein and other embodiments may be used in systems other than cryotherapy systems, where it is desirable to eliminate the gap-space between a delivery catheter and endoscope working channel in order to inhibit and/or remove build-up and backflow of undesired material within the working channel.

As used herein, the singular forms “a,” “an,” and “the” are intended to include the plural forms as well, unless the context clearly indicates otherwise. It will be further understood that the terms “comprises” and/or “comprising,” or “includes” and/or “including” when used herein, specify the presence of stated features, regions, steps elements and/or components, but do not preclude the presence or addition of one or more other features, regions, integers, steps, operations, elements, components and/or groups thereof.

As used herein, the term “distal” refers to the end farthest away from the medical professional when introducing a device into a patient, while the term “proximal” refers to the end closest to the medical professional when introducing a device into a patient.

In various of the described and other embodiments, the present disclosure relates to a cryotherapy system that inhibits or removes the accumulation or buildup and backflow of cryogen spray and other undesired materials, gases and liquids during a cryotherapy medical procedure from within the working channel of an endoscope. Exemplary cryotherapy systems in which the present disclosure may be implemented include, but are not limited to, those systems described in commonly owned U.S. Pat. Nos. 9,820,797, 9,301,796, and 9,144,449, and U.S. patent application Ser. Nos. 11/956,890, 12/022,013, and 14/012,320 each of which are herein incorporated by reference in their entirety.

A typical cryotherapy procedure may involve advancing an endoscope into a body lumen of a patient such that a distal end of the endoscope is positioned in the vicinity of a pre-determined treatment area. A cryogen delivery catheter may then be advanced through a working channel of the endoscope such that a distal tip (e.g., nozzle, etc.) of the cryogen delivery catheter extends distally beyond the endoscope working channel. The cryogen delivery catheter may be fluidly connected to an external cryogen source (e.g., liquid nitrogen) such that cryogen is delivered through the distal tip in the form of cryogen spray which causes target tissue within the treatment area to freeze or “cryofrost.” As discussed herein, the cryotherapy systems of the present disclosure may include a variety of integral or component catheter sleeve designs configured to reduce or eliminate the gap-space between the outer surface of the cryogen delivery catheter and inner surface of the endoscope working channel. The reduction or elimination of the gap-space acts to inhibit or remove the accumulation of undesired materials, such as cryogen spray and other body gases and/or fluids, from either or both of the delivery catheter and working channel.

Referring to FIG. 1, in one embodiment, the present disclosure may include a catheter sleeve 104 formed from a heat-shrink material wrapped around a distal portion of a cryotherapy catheter 102 and directly bonded or affixed thereto by the application of heat. Non-limiting examples of suitable forms of heat-shrink materials may include coatings, tapes or tubes that are applied to the distal portion of the catheter. The materials of the sleeve may be formed from polyethylene and/or polypropylene, or any other suitable polymer materials, as are known in the art. The shape, thickness and length of the catheter sleeve 104 may be adjusted as necessary to form a friction or interference fit with the working channel of an endoscope. The desired thickness of the sleeve, which may be measured as the gap-space between the catheter and the inner surface of the working channel, may be accomplished by one thickness of material or by including multiple wrappings of material in layers prior to the application of heat. The position of the catheter sleeve 104 is not necessarily limited to the configuration illustrated in FIG. 1, as the catheter sleeve may be positioned closer to, or farther away from, the distal end of the cryotherapy catheter. It should also be appreciated that multiple catheter sleeves (e.g., two or more) of the same or different lengths, materials, thicknesses, etc., may be positioned in evenly or unevenly spaced intervals along the distal portion of the cryotherapy catheter. The sleeve may be incorporated onto the distal end of a catheter as an independent manufacturing step after a shaft of the delivery catheter is constructed or the sleeve may be incorporated as part of the construction of the shaft.

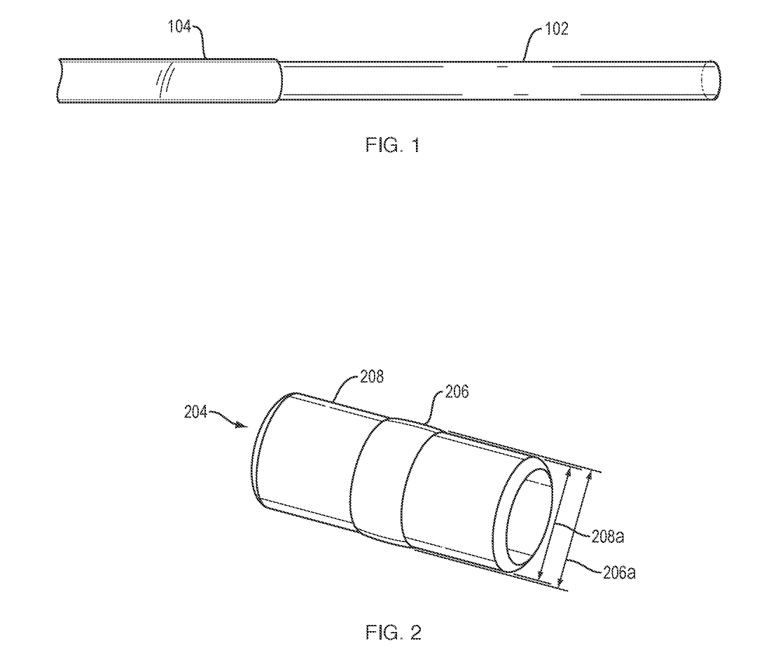

Referring to FIGS. 2-9, in other embodiments, the present disclosure may include a unitary catheter sleeve formed from a variety of flexible, compressible and/or compliant materials, e.g., by a co-extrusion or insert molding process, as are known in the art and applied to the distal end of a delivery catheter. For example, the catheter sleeve may be manufactured from a variety of materials, including, but not limited to, high durometer PET (polyethylene terephthalate) or a more flexibly compliant and lower durometer TPE (thermoplastic elastomer) or TPU (thermoplastic polyurethane). Without intending to limit the scope of the present disclosure, various embodiments of the catheter sleeves disclosed herein generally include a proximal end, a distal end, a lumen defining an inner surface extending between the proximal and distal ends, and an outer surface. The inner surface of the lumen may be configured to slidably receive, and form a friction or interference fit with, an outer surface of a cryogen delivery catheter, such as a cryotherapy catheter. In addition, or alternatively, the catheter sleeve may be permanently bonded or affixed to the cryotherapy catheter by a suitable glue, adhesive or resin.

The outer surface of the catheter sleeve may include a first portion with a first outer diameter and a second portion with a second outer diameter greater than the first outer diameter. The second outer diameter may be equal to or greater than an inner diameter of an endoscope working channel. The “oversized” configuration of the second outer diameter of the catheter sleeve relative to the endoscope working channel may allow the catheter sleeve to compress within the endoscope working channel, thereby forming a seal which spans and closes or substantially closes the gap-space between the catheter and the inner surface of the working channel in order to inhibit or remove the accumulation or backflow of undesired materials, including cryogen spray, from entering or building-up in the working channel, while the “under-sized” configuration of the first outer diameter minimizes friction with the endoscope working channel such that the cryogen delivery catheter is able to slide (e.g., move proximally and distally) along the length of the working channel. In the various configurations provided below, it should be appreciated that the larger outer diameter of the second portion may provide a cleaning or “squeegee” effect as the catheter sleeve slides through the endoscope working channel. Once the catheter sleeve is in position at or near a distal end of the endoscope, the larger outer diameter second portion may also act as a stopper which closes the gap-space between the outer surface of the cryogen delivery catheter and inner surface of the endoscope working channel to block cryogen spray and other body gases and liquids from entering the working channel altogether.

For example, an endoscope working channel may include an inner diameter of approximately 2.0 mm to approximately 3.8 mm, and a second outer diameter of the catheter sleeve may include an outer diameter approximately 0-30% larger than the endoscope working channel (e.g., approximately 2.0 mm to approximately 4.9 mm with the exact dimension chosen for the specific working channel diameters). In addition, or alternatively, if a distal portion of the endoscope working channel is tapered to a smaller diameter at the distal end, the sleeve may be configured to accommodate the taper, thereby providing a seal between the sleeve and the tapered region of the working channel at the distal end of the cryotherapy system while allowing the catheter sleeve to move (e.g., slide) more freely through the remaining portion of the working channel. The ability of the catheter sleeve to slide more easily through the working channel, while maintaining the seal against the working channel. may be further enhanced by coating the catheter sleeve with a lubricious material, including, by way of non-limiting example, a parylene or silicone coating. The various embodiments of the catheter sleeves disclosed herein are non-limiting examples, and any other sizes, shapes, number, orientations and/or configurations of sleeves which span and close or substantially closes the gap-space between the catheter and the inner surface of the working channel to inhibit or remove the accumulation or backflow of undesired materials, including cryogen spray, from entering or building-up in the working channel are within the scope of the present disclosure.

Disclosed embodiments do not necessarily have to be unitarily formed, but may include, e.g., a central member (possibly formed from non-compliant materials such as metal, ceramic, hard plastics etc.) with the outer diameter second portion comprising the compressible material attached thereto. For example, the outer diameter second portion may be formed from a compressible material and may be attached to the outer surface of the sleeve to provide the squeegee and blocking functions.

In reference to FIG. 2, an embodiment of the present disclosure may include a catheter sleeve 204 comprising a first portion 208 with a first outer diameter 208a, and an elevated second portion 206 with a second outer diameter 206a formed as a radial band. As discussed above, the radial band may compress within the working channel of the endoscope to close the gap-space and prevent cryogen spray accumulation, while also clearing the working channel of undesired materials (e.g., accumulated gases, body fluids, and/or particulate matter). Both or either end of the band may include a taper to help keep the sleeve from getting lodged in the working channel. The band may have a uniform flat surface, feature one or more curves, or feature a curve at each end that converge to an apex point.

In reference to FIG. 3, an embodiment of the present disclosure may include a catheter sleeve 304 comprising a first portion 308 with a first outer diameter 308a, and an elevated second portion 306 with a second outer diameter 306a formed as one or more radial V-channel ribs. The one or more radial V-channel ribs may compress within the working channel of the endoscope to close the gap-space, while the channels also provide opposing flexible edges to inhibit or remove accumulation or build-up and backflow of cryogen spray and other undesired materials, gases and liquids from within the working channel of the endoscope. Other configurations of channels are possible, such as C-shaped, U-shaped, W-shaped, etc., as long as the flexible edges or surfaces compress within the working channel of the endoscope to close the gap-space and inhibit or remove, e.g., cryogen spray accumulation. Multiple V-channel or other channeled ribs may provide the advantage of redundant elevated surfaces to improve removal of unwanted materials, although a single channeled rib may function sufficiently as well.

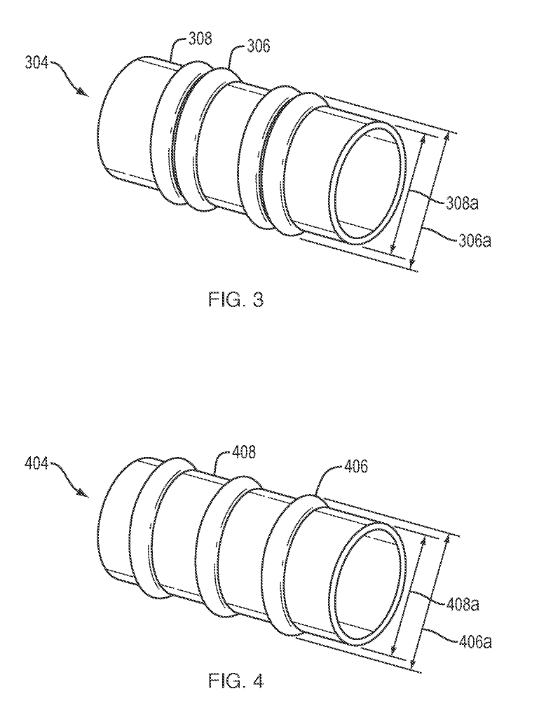

In reference to FIG. 4, an embodiment of the present disclosure may include a catheter sleeve 404 comprising a first portion 408 with a first outer diameter 408a, and an elevated second portion 406 with a second outer diameter 406a formed as one or more radial ribs. The one or more radial ribs may be spaced evenly or staggered along the sleeve 404. Rib shapes may include various geometries, for example, trapezoids, triangles, splined curves, etc. The one or more radial ribs may compress within the working channel of the endoscope to close the gap-space and to inhibit or remove accumulation or build-up and backflow of cryogen spray and other undesired materials, gases and liquids within the working channel of the endoscope. Multiple radial ribs may provide redundant elevated surfaces to ensure complete removal of unwanted materials.

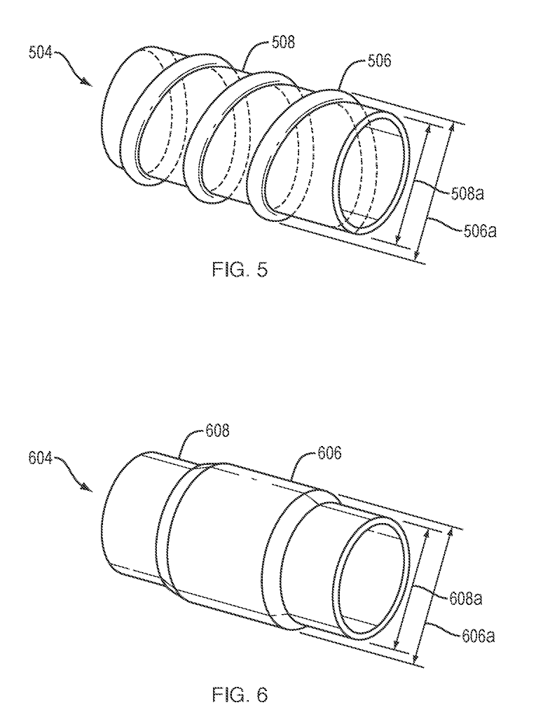

In reference to FIG. 5, an embodiment of the present disclosure may include a catheter sleeve 504 comprising a first portion 508 with a first outer diameter 508a, and an elevated second portion 506 with a second outer diameter 506a formed as a radial spiral rib. The radial spiral rib may compress within the working channel of the endoscope to close the gap-space and to inhibit or remove accumulation or build-up and backflow of cryogen spray and other undesired materials, gases and liquids within the working channel of the endoscope. Rib shapes may include various geometries, for example, trapezoids, triangles, splined curves, etc. Rib designs may vary in pitch and number of revolutions in the spiral along the sleeve. The spiral design may facilitate smoother sliding of the catheter through the endoscope working channel by rotating the cryogen delivery catheter (e.g., clockwise or counter-clockwise) while the cryogen delivery catheter is being advanced or retracted.

In reference to FIG. 6, an embodiment of the present disclosure may include a catheter sleeve 604 comprising a first portion 608 with a first outer diameter 608a, and an elevated second portion 606 with a second outer diameter 606a formed as a radial elongate band. The band may taper at either or both ends from the second portion 606 to the first portion 608. The second portion 606 may be a uniform second portion between two tapering end portions. The radial elongate band may compress within the working channel of the endoscope to close the gap-space and to inhibit or remove accumulation or build-up and backflow of cryogen spray and other undesired materials, gases and liquids within the working channel of the endoscope. The larger surface area of the radial elongate band, as compared to the radial band 206 of FIG. 2, may provide a stronger and more robust plug to close the gap-space.

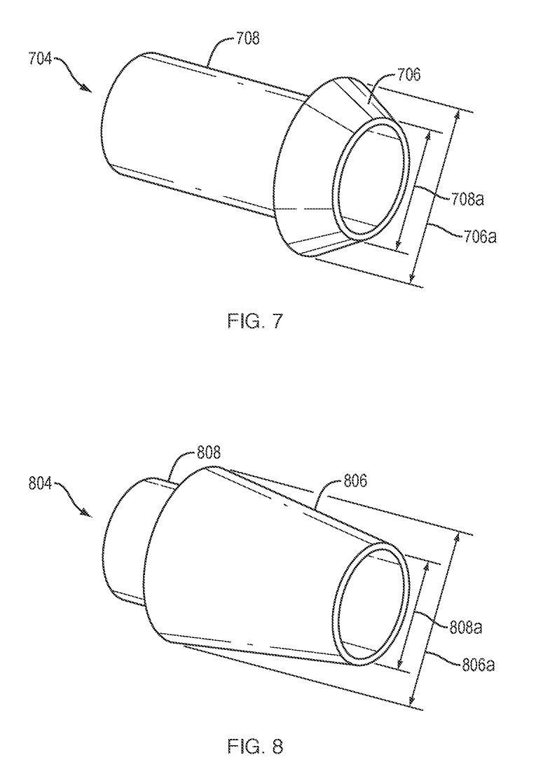

In reference to FIG. 7, an embodiment of the present disclosure may include a catheter sleeve 704 comprising a first portion 708 with a first outer diameter 708a, and an elevated second portion 706 with a second outer diameter 706a formed as a radial flared end. The radial flared end may have an outer diameter that is wider at the proximal end of the catheter sleeve 704 and gradually narrows (e.g., tapers) toward the distal end of the catheter sleeve. The radial flared end may include multiple flared peaks along the body of the band that may be spaced apart from each other. The radial flared end may compress within the working channel of the endoscope to close the gap-space and to inhibit or remove accumulation or build-up and backflow of cryogen spray and other undesired materials, gases and liquids within the working channel of the endoscope

In reference to FIG. 8, an embodiment of the present disclosure may include a catheter sleeve 804 comprising a first portion 808 with a first outer diameter 808a, and an elevated second portion 806 with a second outer diameter 806a formed as a radial tapered flare. The radial tapered flare may have an outer diameter that is wider at the proximal end of the catheter sleeve and gradually narrows (e.g., tapers) toward the distal end of the catheter sleeve in a long and gradual slope to form the shape of a “cork.” The narrowed distal end of the radial tapered flare may allow the catheter sleeve to fit within the endoscope working channel and compress with incrementally increasing friction to close the gap-space and to inhibit or remove accumulation or build-up and backflow of cryogen spray and other undesired materials, gases and liquids within the working channel of the endoscope.

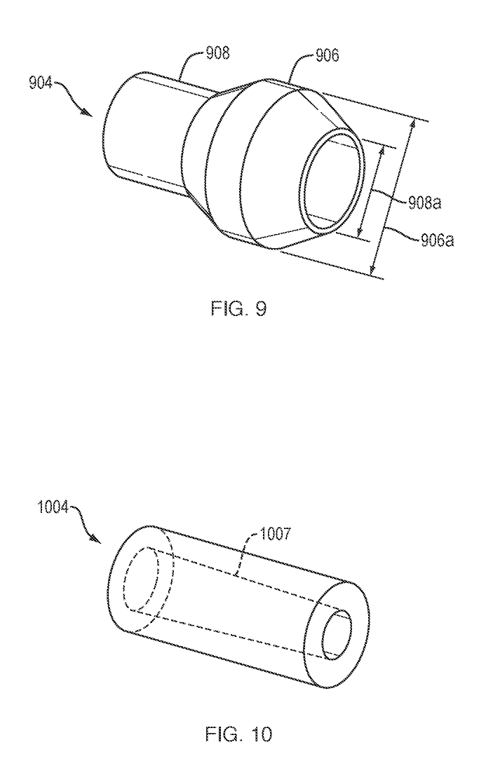

In reference to FIG. 9, an embodiment of the present disclosure may include a catheter sleeve 904 comprising a first portion 908 with a first outer diameter 908a, and an elevated second portion 906 with a second outer diameter 906a formed as a radial band with sloped proximal and distal surfaces. The proximal and distal ends of the band may include a sloped surface which tapers away from the portion of the band with the second outer diameter 908a. The band may compress within the working channel of the endoscope to close the gap-space and to inhibit or remove accumulation or build-up and backflow of cryogen spray and other undesired materials, gases and liquids within the working channel of the endoscope.

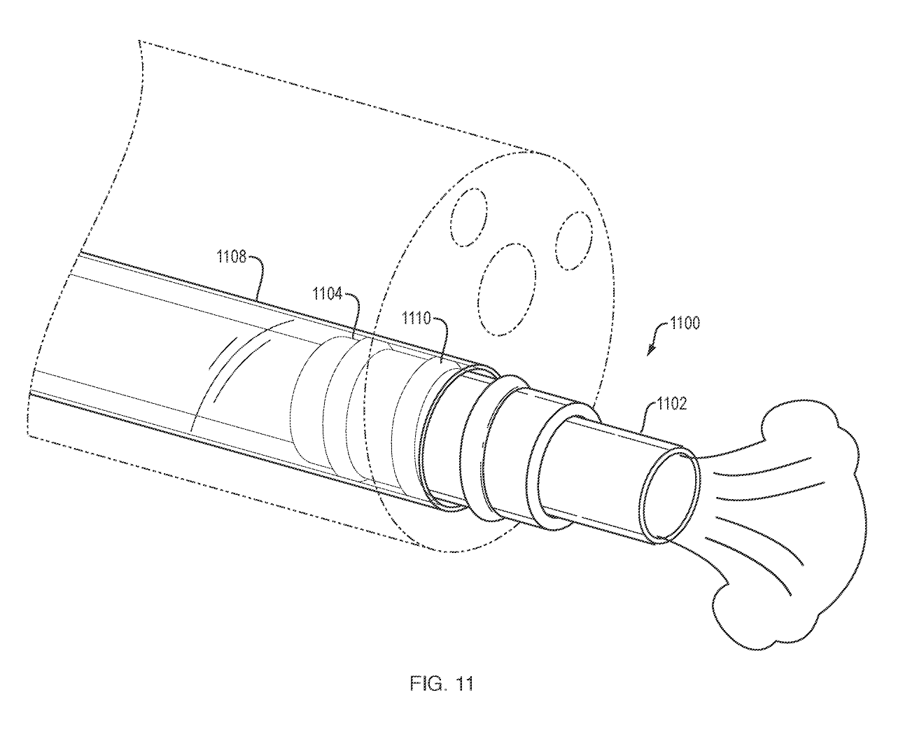

In reference to FIG. 10, an embodiment of the present disclosure may include a catheter sleeve 1004 comprising a lumen 1007 that is wider at the proximal end of the catheter sleeve and gradually narrows (e.g., tapers) toward the distal end. The tapered lumen 1007 may allow the catheter sleeve to slide over the cryogen delivery catheter with less friction than a constant-diameter lumen, while providing a sufficiently strong interference fit to maintain proper positioning of the catheter sleeve on the cryogen delivery catheter during the cryotherapy procedure. In addition, or alternatively, the tapered lumen 1007 may accommodate cryogen delivery catheters having a variety of shapes and/or sizes. Although the outer surface of the catheter sleeve 1004 includes a smooth outer surface, it should be appreciated that any of the catheter sleeve configurations discussed above, and other embodiments of a sleeve having an elevated larger diameter portion, may include a tapered lumen. The catheter sleeve 1004 ends may include an inner or outer radius to facilitate advancement and retraction within the tapered lumen 1007 and/or a working channel of an endoscope.

In reference to FIG. 11, an embodiment of the present disclosure may include a cryotherapy system 1100 comprising a cryogen delivery catheter 1102, a catheter sleeve 1104, and an endoscope working channel 1108. Although the cryotherapy system 1100 of FIG. 11 depicts a catheter sleeve as illustrated in FIG. 4, any of the catheter sleeve configurations disclosed herein, and other embodiments of a sleeve having an elevated larger diameter portion, may be suitable for use on a delivery catheter disposed within the endoscope working channel. The radial ribs which form the elevated second portion 1110 of the catheter sleeve 1104 may compress against the inner surface of the endoscope working channel 1108 to close the gap-space and prevent cryogen spray accumulation.

A cryotherapy procedure using a delivery catheter with a sleeve within an endoscope, in accordance with systems and methods of the present disclosure may include inserting a catheter into the working channel of the endoscope with a distal end portion having a sleeve section. The sleeve section may have an outer surface, the outer surface may include at least one first portion with a first diameter and at least one second portion with a second diameter that is greater than the first diameter and that is dimensioned to contact an inner surface of an endoscope working channel. The sleeve section may be kept at a distal end of the working channel of the endoscope, to close the gap-space and to inhibit or remove accumulation or build-up and backflow of cryogen spray and other undesired materials, gases and liquids within the working channel of the endoscope. While the second diameter of the sleeve is in contact with the inner diameter of the working channel of the endoscope, the catheter may be translated (e.g. by sliding) distally and/or proximally within the working channel of the endoscope to remove undesired materials from the working channel. A method may include shielding at least one sensor mounted on the catheter or within the working channel of the endoscope proximal to the sleeve section. The sleeve section may prevent undesirable fluids from traveling proximally into the working channel. Without shielded protection, undesirable thermal effects due to fluid or gas buildup within the working channel could result. The shielding effect could mitigate these undesirable effects. The method may include eliminating undesired gases or fluids from the working channel of the endoscope to minimize negative thermal effects on the strength of cryospray during a cryotherapy procedure. This may be achieved through shielding or by translating the sleeve section through the working channel, forcing fluids or gases out.

All of the devices and/or methods disclosed and claimed herein can be made without undue experimentation in light of the present disclosure. While the devices and methods of this disclosure have been described in terms of preferred embodiments, it may be apparent to those of skill in the art that variations can be applied to the devices and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

What is claimed is:

1. A cryogen delivery catheter, comprising:
an elongate shaft having a distal end portion, and
a sleeve section comprising an outer surface, the outer surface including at least one first portion with a first diameter and at least one second portion with a second diameter that is greater than the first diameter and that is dimensioned to contact an inner surface of an endoscope working channel.

2. The catheter of claim 1, wherein the sleeve section is a tubular member defining a lumen.

3. The catheter of claim 1, wherein the sleeve section is integral with the shaft.

4. The catheter of claim 1, wherein the first diameter is the same as the outer diameter of the catheter shaft.

5. The catheter of claim 1, wherein the second portion includes a radial mid-body band.

6. The catheter of claim 1, wherein the second portion includes a tapered incline with a slope facing distally.

7. The catheter of claim 1, wherein the second portion includes a planar middle portion that tapers in diameter at both ends to the first diameter of the first portion.

8. The catheter of claim 1, wherein the sleeve section includes a radial band with a sloped proximal surface and a sloped distal surface.

9. The catheter of claim 1, wherein the sleeve section includes a lumen with a tapered diameter.

10. A cryotherapy system, comprising:
an endoscope;
a cryogen delivery catheter slidably disposed within a working channel of the endoscope; and
a catheter sleeve disposed around a distal portion of the cryogen delivery catheter, the catheter sleeve including an outer surface having a first diameter portion and a second diameter portion, the second diameter portion having a greater diameter than the first diameter portion and configured to contact an inner wall of the working channel.

11. The cryotherapy system of claim 10, wherein the second diameter portion is configured to span a gap-space between the distal portion of the cryogen delivery catheter and the working channel of the endoscope.

12. The cryotherapy system of claim 10, wherein the catheter sleeve forms an interference fit with the distal portion of the cryogen delivery catheter.

13. The cryotherapy system of claim 10, wherein the catheter sleeve is adhered to the distal portion of the cryogen delivery catheter.

14. The cryotherapy system of claim 10, wherein the diameter of the second diameter portion is greater than or equal to a diameter of the working channel of the endoscope.

15. The cryotherapy system of claim 10, wherein the second diameter portion is configured to remove or inhibit build-up of undesired material from the working channel of the endoscope as the delivery catheter slides distally within the working channel.

16. The cryotherapy system of claim 10, wherein the catheter sleeve is integrally formed as a part of the distal portion of cryogen delivery catheter.

17. A method of maintaining a working channel of an endoscope comprising:
inserting a catheter into the working channel of the endoscope with a distal end portion having a sleeve section comprising an outer surface, the outer surface including at least one first portion with a first diameter and at least one second portion with a second diameter that is greater than the first diameter and that is dimensioned to contact an inner surface of the working channel of the endoscope.

18. The method of claim 17, further comprising translating the catheter within the working channel of the endoscope.

19. The method of claim 17, further comprising shielding at least one sensor mounted on the catheter or within the working channel of the endoscope proximal to the sleeve section, whereby undesired thermal effects due to gases or fluids build up within the working channel are mitigated.

20. The method of claim 17, further comprising eliminating undesired gases or fluids from the working channel of the endoscope, whereby negative thermal effects on the strength of cryospray during a cryotherapy procedure are minimized.
